(19) United States
(12) Patent Application Publication
(10) Pub. No.: US 2025/0378908 A1
(43) Pub. Date: Dec. 11, 2025

(54) IDENTIFYING SOMATIC PSEUDOGENES AS A PROXY FOR RESTROTRANSPOSITION ACTIVITY DETECTION

(71) Applicant: GUARDANT HEALTH, INC., Palo Alto, CA (US)

(72) Inventors: Vitaly BALAN, Foster City, CA (US); Aaron Isaac HARDIN, San Francisco, CA (US); James HART, Sunnyvale, CA (US); Sofia MEDINA RUIZ, Palo Alto, CA (US)

(21) Appl. No.: 18/737,816

(22) Filed: Jun. 7, 2024

Related U.S. Application Data
(60) Provisional application No. 63/506,880, filed on Jun. 8, 2023.

(51) Int. Cl. G16B 20/20; G16B 30/10; G16B 45/00; G16H 40/67

(52) U.S. Cl. CPC G16B 20/20; G16B 30/10; G16B 45/00; G16H 40/67

(57) ABSTRACT

Described herein is a method for detecting pseudogenes, including processed pseudogenes, further including detection for measuring retrotrasposon element activity. Such measurements are useful in screening and detecting cancer in subjects, including predicting the likelihood or cancer, recurrence, treatment responsiveness and selection.

### CROSS REFERENCE TO RELATED APPLICATION

The present application claims the benefit of U.S. Provisional Application No. 63/506,880, filed Jun. 8, 2023. Both incorporated by reference in their entirety for all purposes.

### BACKGROUND

Pseudogenes have largely been considered lacking significant functions as a result of the accumulation of mutations, including frameshift, premature stop-codons and relocation of genes to inactive heterochromatin regions of the genome. The two main groups of pseudogenes, processed and unprocessed, are categorized by primary structure and origin. A minority, 10% of all pseudogenes, are transcribed into RNAs and participate in parental gene expression regulation at both transcriptional and translational levels through senseRNA (sRNA) and antisense RNA (asRNA).

Pseudogenes in the different types of cancers could be useful in molecular diagnostics and can be detected in various types of biological material including tissue as well as liquid biopsy. There is a great need in the art to evaluate the role of pseudogenes as involved in the development and progression of diseases such as cancer.

Described herein is the use of pseudogene detection as a proxy for retrotranspotion activity detection. Whereas retrotransposition such as LINE-1 activity is increased in various cancer cell lines and in patient tissues resected from primary tumors, retrotransposition also correlates with increased cancer metastasis. Detection of pseudogenes according to the methods described herein provides a variety of diagnostic and screening techniques for oncology.

### SUMMARY OF THE INVENTION

Described herein is a method comprising: determining a plurality of sequence reads of a target region of a chromosome of a subject, wherein the target region of the chromosome comprises one or more loci; aligning the plurality of sequence reads to a reference genome; subtracting aligned pairs from the plurality of sequence reads to generate a plurality of candidate sequence reads; aligning the plurality of candidates sequences reads to a plurality of known allele

sequences; determining, based on the alignment, for each known allele sequence of the plurality of known allele sequences, a number of sequence reads that aligned to each known allele sequence; extracting unaligned candidate sequence reads; mapping unaligned candidate sequence reads to the reference genome; and determining, based on the unaligned candidate sequence reads that mapped to the reference genome, one or more integration sites present at the one or more loci. In other embodiments, the plurality of known allele sequences comprise a plurality of known retrotransposition elements, such as 5′ truncation, 5′ inversion, 3′ transduction, and/or EN-independent insertions. In other embodiments, the plurality of known allele sequences comprise a plurality of known reference repeats. In other embodiments, only one read of a read pair is aligned to a repeat. In other embodiments, the method includes determining retrotransposition activity based on the one or more integration sites present at the one or more loci. In other embodiments, the method includes obtaining a sample from the subject; and sequencing the sample to obtain the plurality of sequence reads of the target region of the chromosome. In other embodiments, the method includes determining, based on the mapping, for each read of the plurality of sequence reads, one or more integration sites present at the one or more loci. In other embodiments, the target region comprises one or more of the following genes: ADAM5, ACTG1P25, AK4P1, BRAFP1, BRCA1P1, CYP2A7, CYP4Z2P, DUXAP8, EBLN3P, FTH1P3, FLT1P1-S, OGFRP1, LGMNP1, MSTO2P, MYLKP1, OCT4-pg4, PCNAP1, PDIA3P1, PPM1K, PREL1D1P6, PTENP1-AS, PTTG3P, RPSAP52, SALL4P5, TCAM1P, TDGF1P3, RP9P, UBE2CP3, ARHGAP27P1, FLT1P1-AS, FOXO3P, Pseudogenes of FTH1, GUSBP11, MT1JP, PEBP1P2, SNRPFP1, SNX17 and TUSC2P. In other embodiments, the target region comprises one or more of the genes in Table 1.

In other embodiments, the method includes determining one or more integration sites comprises identifying one or more exon-exon junctions in the reference genome. In other embodiments, the one or more exon-exon junctions is not of germline origin. In other embodiments, the method includes determining one or more integration sites comprises mapping to a maximum pseudogene sequence comprising all exons. In other embodiments, the method includes mapping comprises determining unaligned candidate sequence spanning one or more exon-exon junctions.

In other embodiments, the method includes two reads of a read pair is mapped to different exons.

In other embodiments, the method includes determining, based on the numbers of sequence reads that aligned to each known allele sequence, for the one or more loci, the known allele sequences present at the one or more loci comprises determining one or more known allele sequences having a highest number of sequence reads aligned.

In other embodiments, the reads that aligned to each known allele sequence are grouped into read families, and the method further comprises, determining a number of sequence read families that are aligned to each known allele sequence. In other embodiments, the method includes determining, for the one or more loci, the known allele sequences present at the one or more loci based on the numbers of sequence read families that aligned to each known allele sequence. In other embodiments, the method includes determining a length of a portion of each known allele sequence aligned to two or more sequence reads of the plurality of sequence reads. In other embodiments, the method includes sorting, for a locus, the known allele sequences present at the locus by the number of sequence reads that aligned to each known allele sequence;
determining, for the locus, a first known allele sequence with a highest number of
sequence reads aligned; inserting the first known allele sequence with the highest number of sequence reads aligned into a superset; determining one or more known allele sequences that aligned to reads that are a subset of the reads aligned to the first known allele sequence; and
inserting the one or more known allele sequences into the superset. In other embodiments, the superset comprises a graph data structure. In other embodiments, the graph data structure comprises a directed acyclic graph. In other embodiments, the graph data structure represents a Hasse diagram. In other embodiments, the method includes determining that the locus is associated with a single superset; and determining the first known allele sequence of the single superset as the allele present at the locus. In other embodiments, the method includes determining a plurality of supersets for the locus. In other embodiments, the method includes determining, based on the plurality of supersets for the locus, two supersets with a
cumulative largest number of distinct reads; and determining the first known allele sequence of each of the two supersets as the alleles present at the locus. In other embodiments, the method includes assisting in a communication of the known allele sequences present at the one or more loci to a medical provider.

Described herein is a method comprising: determining a plurality of sequence reads of a target region of a chromosome of a subject, wherein the target region of the chromosome comprises one or more loci; aligning the plurality of sequence reads to a reference genome; subtracting aligned pairs from the plurality of sequence reads to generate a plurality of candidate sequence reads; aligning the plurality of candidates sequences reads to a plurality of known allele

sequences; determining, based on the alignment, for each known allele sequence of the plurality of known allele sequences, a number of sequence reads that aligned to each known allele sequence; extracting unaligned candidate sequence reads; mapping unaligned candidate sequence reads to the reference genome; and determining, based on the unaligned candidate sequence reads that mapped to the reference genome, one or more integration sites present at the one or more loci. In other embodiments, the plurality of known allele sequences comprise a plurality of known retrotransposition elements, such as 5′ truncation, 5′ inversion, 3′ transduction, and/or EN-independent insertions. In other embodiments, the plurality of known allele sequences comprise a plurality of known reference repeats, only one read of a read pair is aligned to a repeat, the method includes determining, based on the mapping, for each read of the plurality of sequence reads, one or more integration sites present at the one or more loci, the method includes determining one or more integration sites comprises identifying one or more exon-exon junctions in the reference genome, the one or more exon-exon junctions is not of germline origin.

In other embodiments, the method includes determining one or more integration sites comprises mapping to a maximum pseudogene sequence comprising all exons. In other embodiments, the method includes mapping comprises determining unaligned candidate sequence spanning one or more exon-exon junctions. In other embodiments, the method includes two reads of a read pair is mapped to different exons. In other embodiments, the method includes determining, based on the numbers of sequence reads that aligned to each known allele sequence, for the one or more loci, the known allele sequences present at the one or more loci comprises determining one or more known allele sequences having a highest number of sequence reads aligned. In other embodiments, the method includes determining retrotransposition activity based on the one or more integration sites present at the one or more loci.

Described herein is determining the likelihood of a subject being afflicted with cancer, recurrence of cancer, or responsiveness to therapy for cancer, including determining a plurality of sequence reads of a target region of a chromosome of a subject, wherein the target region of the chromosome comprises one or more loci; aligning the plurality of sequence reads to a reference genome; subtracting aligned pairs from the plurality of sequence reads to generate a plurality of candidate sequence reads; aligning the plurality of candidates sequences reads to a plurality of known allele sequences; determining, based on the alignment, for each known allele sequence of the plurality of known allele sequences, a number of sequence reads that aligned to each known allele sequence; extracting unaligned candidate sequence reads; mapping unaligned candidate sequence reads to the reference genome; and determining, based on the unaligned candidate sequence reads that mapped to the reference genome, one or more integration sites present at the one or more loci.

A system for performing any of the aforementioned methods. A computer readable medium for performing any of the aforementioned methods.

### BRIEF DESCRIPTION OF THE FIGURES

FIG. 1. Reverse transcriptase of LINE1 (RTL1) and how it generated retrobiome

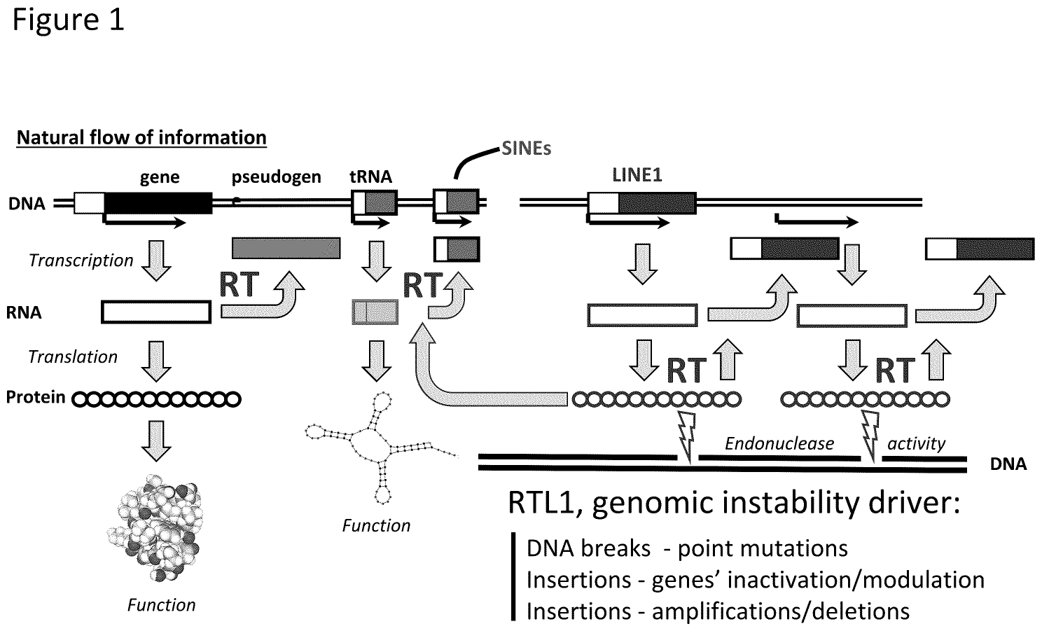

FIG. 2. ORF2 expression in human normal and cancer tissue sections.

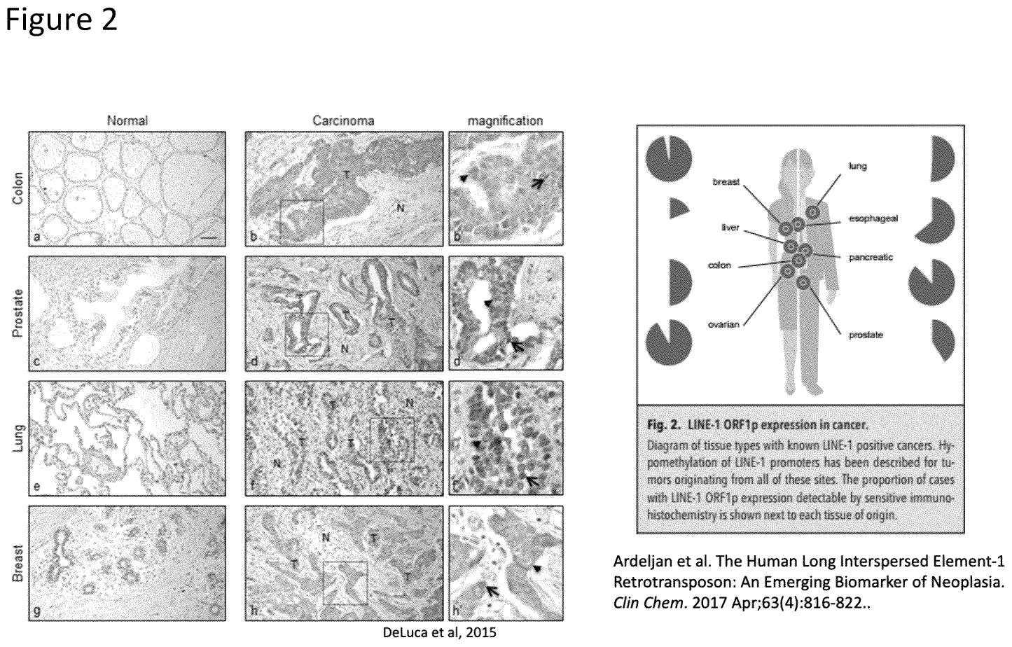

FIG. 3. Detecting cells with active retrotransposon elements (REs).

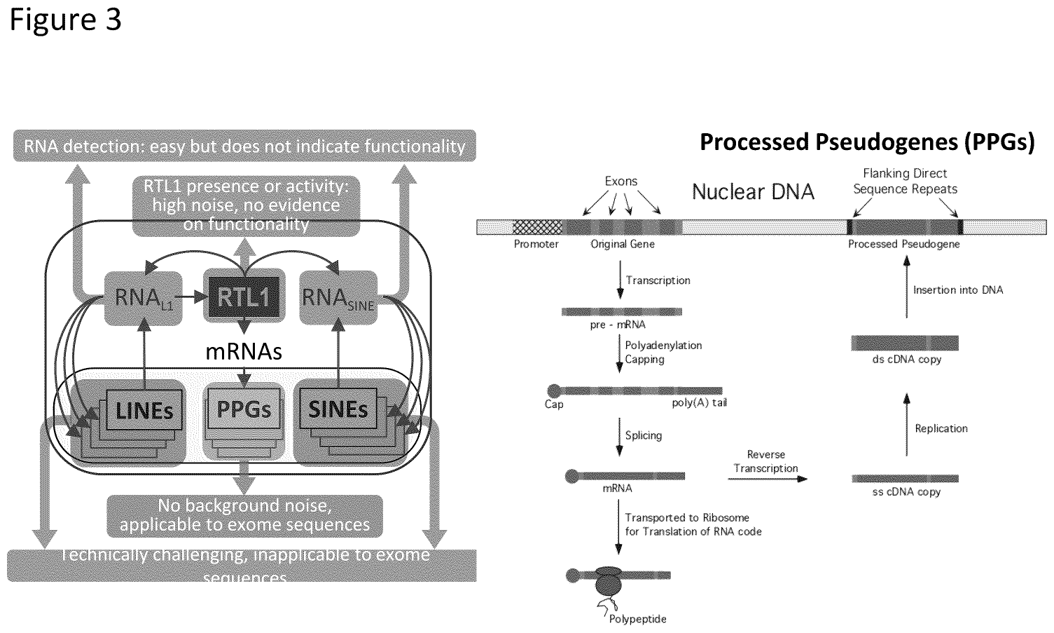

FIG. 4. Processed pseudogenes (PPGs). There are ˜15,000 processed pseudogenes in human genome ranging in copy number between 1-142 (adapted from Ewing et al. (2013)). They originate from ˜5000 of human genes. Somatic cell transcriptome of tumors can give rise to new PPGs.

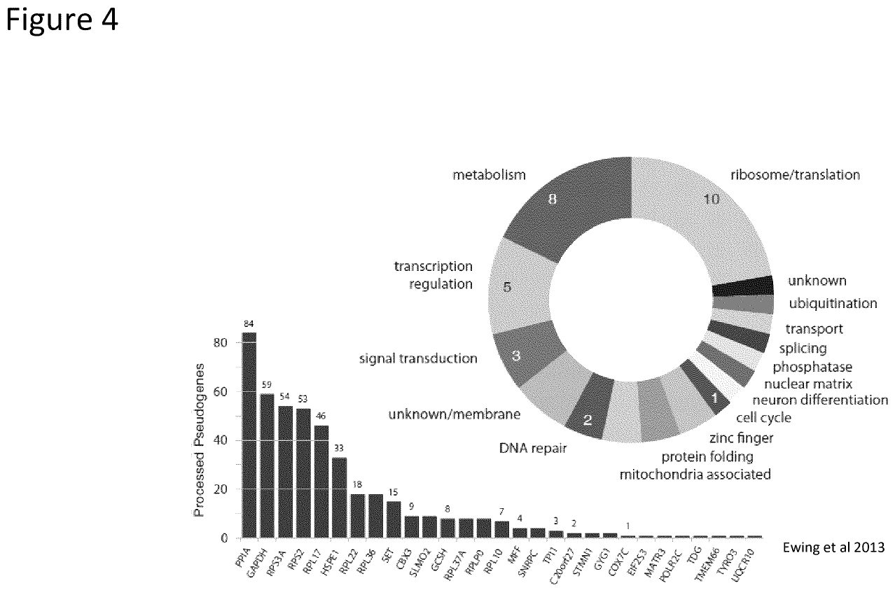

FIG. 5. Computational algorithm for determining PPGs and REs.

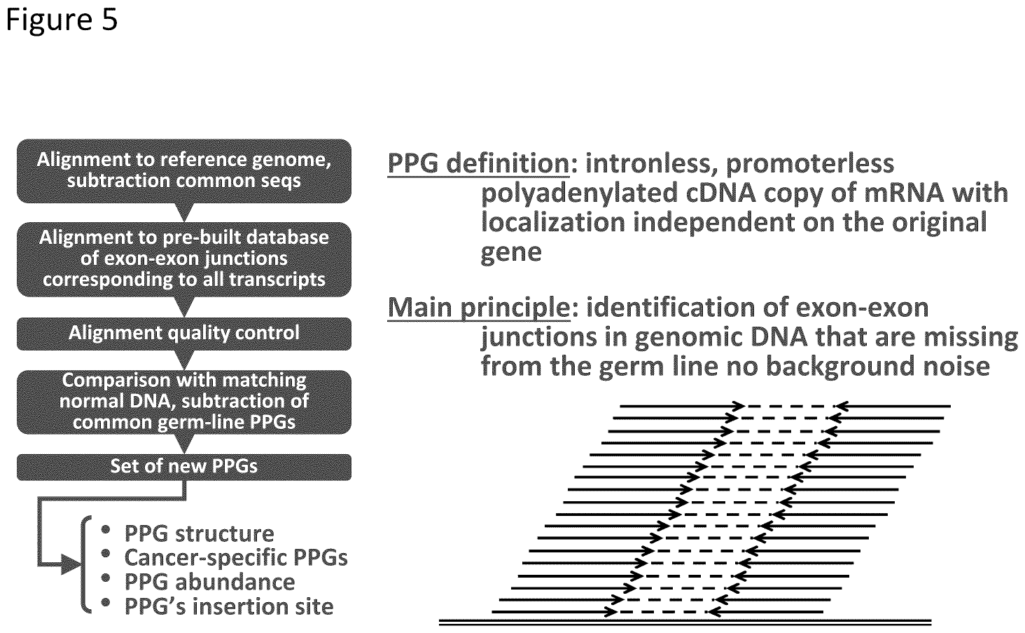

FIG. 6. New PPGs in five bladder cancer samples. Shown is identification in SNX17 (no pseudogenes in reference), In this instance, reads are mapped directly to exon-exon junction and two read in one pair are mapped to different exons.

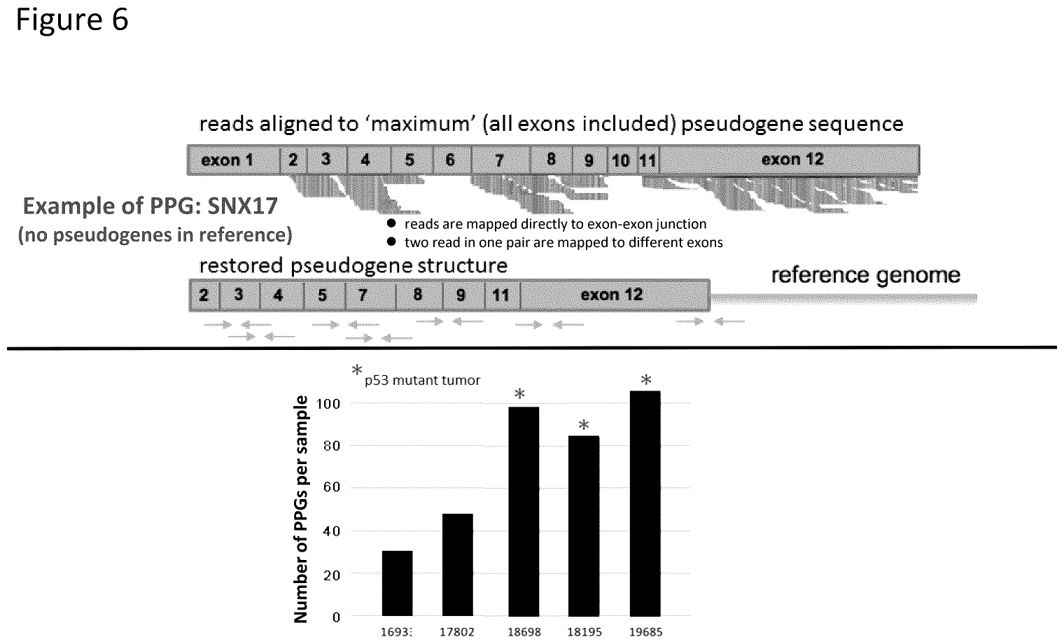

FIG. 7. New retroelements computational algorithm.

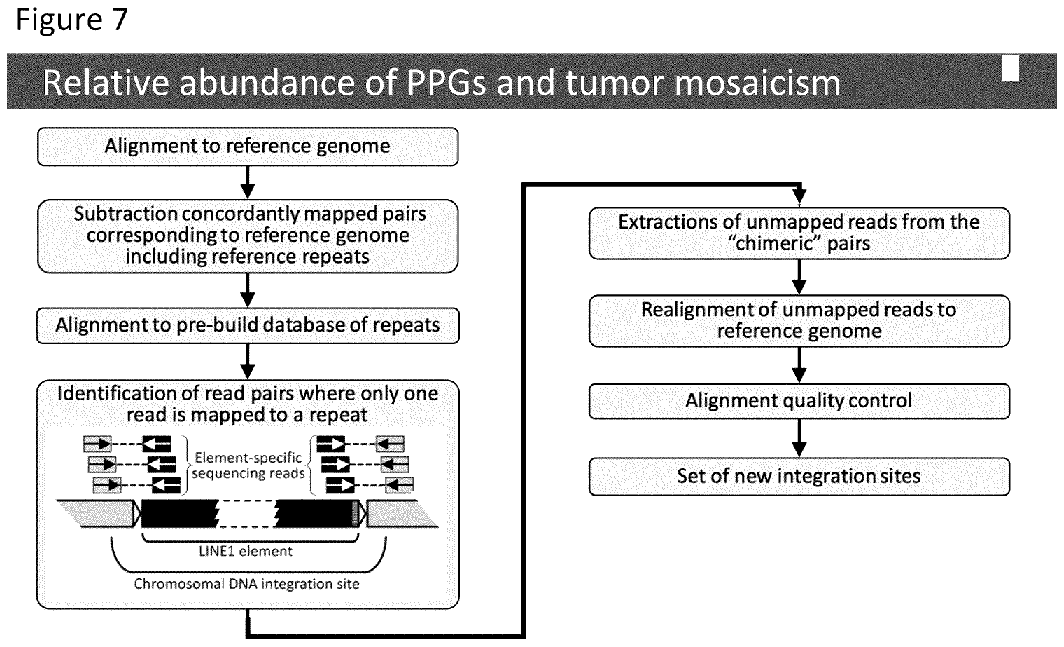

FIG. 8. Pseudogenes are a proxy for RE activity. Shown here are ratios between pseudogenes, LINEs and SINEs.

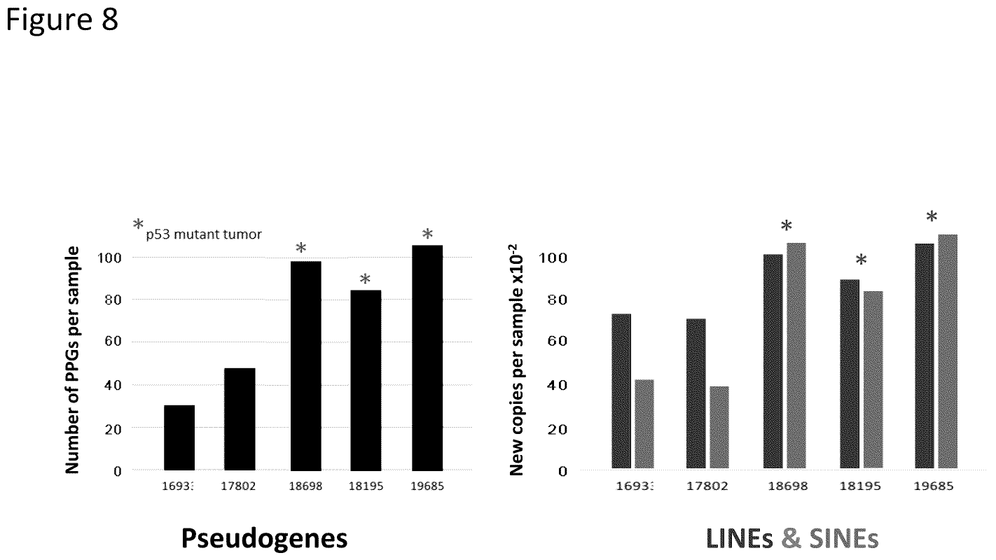

### DETAILED DESCRIPTION

Processed pseudogenes are formed by integration into new genome sites of cDNAs produced by the reverse transcription of parental genes. Due to this reason, processed pseudogenes do not contain introns. Most of these molecules have a poly (A) sequence at the 3′end due to the mRNA 3′end polyadenylation process. Additionally, processed pseudogenes are flanked by duplicated integration sites 5 to 20 bp in length. Unprocessed pseudogenes, contain introns and can be unitary (orphan) or duplicated. Unitary pseudogenes are derived from single-copy functional genes, which accumulated spontaneous mutations during evolution and have lost their primary functions. Therefore, unitary pseudogenes have no paralogs in the same genome but may have orthologs in the relative species. Duplicated pseudogenes arise from tandem duplications of genes during an unequal crossing-over process. The duplicated gene can undergo further mutations, which convert it into a completely new pseudogene. Because of the mechanism of origin, duplicated pseudogenes are situated on the same chromosomes as their parental genes.

A first functional level is interaction and regulation of RNAs molecules. 10% of all pseudogenes are transcribed into RNAs (psRNAs) that participate in the regulation of parental gene expression at both transcriptional and translational levels through senseRNA (sRNA) and antisense RNA (asRNA). sRNA regulates the expression of their parental gene mRNA through competition for miRNA. Due to the significant similarity, they share miRNA binding sites, whose binding to miRNAs ensures the regulatory functions of these RNA molecules in both the nucleus and the cytoplasm. Higher pseudogene transcription activity leads to a higher number of miRNA molecules that bind to its sRNA, which depletes their intracellular pool and reduces suppression of the parental gene expression.

Another function of pseudogenes is generation of long non-coding RNAs (lncRNAs) without protein products. But in some cases, short peptides are generated. lncRNAs function as regulators of transcription by activation of specific genes, modulators of protein factors and chromatin, guides for specific ribonucleoprotein complexes as well as scaffolds for specified ribonucleoproteins. It is also postulated that lncRNAs function as molecular sponges for miRNA. lncRNAs could probably be used as biomarkers in oncology.

The second type of regulation is the ability to modulate DNA, which is manifested by random insertion of a pseudogene sequence into the parental or other host gene as well as causing DNA sequence exchange between the pseudogene and parental gene. The insertion of pseudogene sequence can cause different biological effects: (i) epigenetic silencing, (ii) initiation of transcription, (iii) genetic fusion, or even (vi) mutagenesis. These modifications induce changes in expression level of specific genes or cause alternative functions of them, which could induce carcinogenesis. Another possibility is exchanging DNA sequences between the pseudogene and parental gene. In this case, the conversion as well as recombination is possible. One of the examples of this is the rearrangements between the BRCA1 gene and BRCA1 pseudogene that causes origin of mutated alleles, which lack promoter, are changes in the exons and lack the initiation codon. Exchanging DNA sequences between pseudogene and parental gene strongly influences the genome and could lead to inactivation of suppressor genes or activation of oncogenes.

The last pseudogene function is the possibility of influencing the genome and transcriptome by protein or peptide. Paradoxically, some pseudogenes such as some lncRNAs have open reading frames and encode proteins or peptides and these products could play a regulative function in a cell. These pseudo-proteins or -peptides could have parental gene-like or -unlike functions, cooperate with parental genes or even activate immune response.

As pseudogenes can interact in various ways with DNA, RNA, and proteins participating in the modulation of target gene expression, particularly their parental genes and other epigenetic mechanisms. Genomic outcomes include 5′ truncation, 5′ inversion, 3′ transduction, and/or EN-independent insertions. Therefore, these molecules are involved in the development, and progression of certain diseases, especially cancer.

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed of being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation, pseudogenes, number of pseudogenes, retrotransposon activity, or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

Described are methods including determining a plurality pseudogene sequences, determining a plurality of sequence reads of a target region of a chromosome of a subject, wherein the target region of the chromosome comprises one or more loci, aligning the plurality of sequence reads to the plurality of known or suspected pseudogene allele sequences, determining, based on the alignment, for each known or suspected pseudogene allele sequence of the plurality of known or suspected pseudogene allele sequences, a number of sequence reads that aligned to each known or suspected pseudogene allele sequence, and determining, based on the numbers of sequence reads that aligned to each known pseudogene or suspected allele sequence, for the one or more loci, the known allele sequences present at the one or more loci. Further, the method includes determining retrotransposition activity based on the known allele sequences present at the one or more loci.

Described are methods including determining a plurality of pseudogene allele sequences by determining a plurality of sequence reads of a target region of a chromosome of a subject, wherein the target region of the chromosome comprises one or more loci including known or suspected psudeogene allele sequences, aligning the plurality of sequence reads to the plurality of known pseudogene allele sequences, determining, based on the alignment, for each known or suspected psudeogene allele sequence of the plurality of known or suspected pseudogene allele sequences, a number of sequence read families (i.e., number of nucleic acid molecules—a sequence read family may be a group of sequence reads corresponding to a single nucleic acid molecule) that aligned to each known pseudogene allele sequence, and determining, based on the numbers of sequence read families that aligned to each known pseudogene allele. Further, the method includes determining retrotransposition activity based on the known allele sequences present at the one or more loci. Pseudogene detection relates to other highly conserved genomic segments such as HLA, KIR, etc. Examples of such detection techniques including PCT App. No. PCT/US23/65469 and U.S. Prov. App. No. 63/494,724, each of which is incorporated by reference herein. Described herein are method including determining a plurality of sequence reads of a target region of a chromosome of a subject, wherein the target region of the chromosome comprises one or more loci, subtracting concordantly mapped pairs corresponding to reference genome including reference repeats, aligning to pre-built database of repeats, identifying read pairs where only one read is mapped to a repeated, extracting unmapped reads, realigning unmapped reads to reference genome, identifying sets of new integration sites. Further, the method includes determining retrotransposition activity based on the known allele sequences present at the one or more loci.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, pseudogenes, number of pseudogenes, retrotransposon activity, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation, pseudogenes, number of pseudogenes, retrotransposon activity, or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation, pseudogenes, number of pseudogenes, retrotransposon activity, detection and certain immune states may be monitored. In this example, copy number variation, pseudogenes, number of pseudogenes, retrotransposon activity, analysis may be performed over time to produce a profile of how a particular disease may be progressing. Copy number variation, pseudogenes, number of pseudogenes, retrotransposon activity, or even rare mutation detection may be used to determine how a population of pathogens is changing during the course of infection. This may be particularly important during chronic infections, such as HIV/AIDS or Hepatitis infections, whereby viruses may change life cycle state and/or mutate into more virulent forms during the course of infection.

The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation, pseudogenes, number of pseudogenes, retrotransposon activity, and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site

The present methods can be used to generate or profile, fingerprint or set of data that is a summation of genetic information derived from different cells in a heterogeneous disease. This set of data may comprise copy number variation, pseudogenes, number of pseudogenes, retrotransposon activity, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Compositions Including Captured DNA

Provided herein is a combination including first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population comprises a sequence tag selected from a first set of one or more sequence tags and the second population comprises a sequence tag selected from a second set of one or more sequence tags, and the second set of sequence tags is different from the first set of sequence tags. The sequence tags may comprise barcodes.

In some embodiments, the first population comprises protected hmC, such as glucosylated hmC. In some embodiments, the first population was subjected to any of the conversion procedures discussed herein, such as bisulfite conversion, Ox-BS conversion, TAB conversion, ACE conversion, TAP conversion, TAPSB conversion, or CAP conversion. In some embodiments, the first population was subjected to protection of hmC followed by deamination of mC and/or C. In some embodiments of the combination, the first population comprises or was derived from DNA with a cytosine modification in a greater proportion than the second population and the first population comprises first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to β-6-azide-glucosyl-5-hydroxymethylcytosine that comprises an affinity label (e.g., biotin).

In any of the combinations described herein, the captured DNA may comprise cfDNA. The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set comprises sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems

Methods of the present disclosure can be implemented using, or with the aid of, computer systems. For example, such methods may comprise: partitioning the sample into a plurality of subsamples, including a first subsample and a second subsample, wherein the first subsample comprises DNA with a cytosine modification in a greater proportion than the second subsample;

subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets comprises a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### Cancer and Other Diseases

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation, pseudogenes, number of pseudogenes, retrotransposon activity, or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, pseudogenes, number of pseudogenes, retrotransposon activity, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation, pseudogenes, number of pseudogenes, retrotransposon activity, and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, pseudogenes, number of pseudogenes, retrotransposon activity, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the test subject. Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the test subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a test subject and/or classifying a test subject as being a candidate for a subsequent cancer treatment.

Methods of Determining a Risk of Cancer Recurrence in a Test Subject and/or Classifying a Test Subject as being a Candidate for a Subsequent Cancer Treatment.

In some embodiments, a method provided herein is a method of determining a risk of cancer recurrence in a test subject. In some embodiments, a method provided herein is a method of classifying a test subject as being a candidate for a subsequent cancer treatment.

Any of such methods may comprise collecting DNA (e.g., originating or derived from a tumor cell) from the test subject diagnosed with the cancer at one or more preselected timepoints following one or more previous cancer treatments to the test subject. The subject may be any of the subjects described herein. The DNA may be cfDNA. The DNA may be obtained from a tissue sample.

Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein. In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a test subject may comprise determining a cancer recurrence score that is indicative of the presence or absence, or amount, of the DNA originating or derived from the tumor cell for the test subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a test subject as being a candidate for a subsequent cancer treatment may comprise comparing the cancer recurrence score of the test subject with a predetermined cancer recurrence threshold, thereby classifying the test subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the test subject based on the cancer recurrence score; for example, the DFS period may be 1 year, 2 years, 3, years, 4 years, 5 years, or 10 years.

In some embodiments, the set of sequence information comprises sequence-variable target region sequences, and determining the cancer recurrence score may comprise determining at least a first subscore indicative of the amount of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the test subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the epigenetic target regions, e.g., including one or both of hypermethylation variable target regions and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules including alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10-11 to 1 or 10-10 to 1 is sufficient for the cancer recurrence score to be classified as positive for cancer recurrence. In some embodiments, a fraction of tumor DNA greater than or equal to a threshold in the range of 10-10 to 10-9, 10-9 to 10-8, 10-8 to 10-7, 10-7 to 10-6, 10-6 to 10-5, 10-5 to 10-4, 10-4 to 10-3, 10-3 to 10-2, or 10-2 to 10-1 is sufficient for the cancer recurrence score to be classified as positive for cancer recurrence. In some embodiments, the fraction of tumor DNA greater than a threshold of at least 10-7 is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the first and second subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Cancer Treatments, Therapies

In some cases, the cancer treatment includes, without limitation, imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, BMS-690514, BMS-754807, EMD 525797, GDC-0973, GDC-0941, MK-2206, AZD6244, GSK1120212, PX-866, XL821, IMC-A12, MM-121, PF-02341066, RG7160, and Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and cetuximab.

It has been reported that treating colon cancer cells and tumor organoids with another derivative of a hypomethylating agent (5-aza-2′-deoxycytidine) was sufficient to induce a growth-inhibiting immune response by triggering retrotransposon expression. Combinations of DNMTi and HDACi selectively induced LTR retrotransposons more efficiently than using each drug individually. Baxevanis. “The Regulation and Immune Signature of Retrotransposons in Cancer” Cancers (Basel). 2023 September; 15 (17): 4340. In other embodiments, the described methods and compositions can be applied to cancer therapies with a mechanism of action known to clearly impact methylation. Such therapies include HDAC inhibitors such as vorinostat, romidepsin, panobinostat, chidamide and belinostat HAT inhibitors such as inhibitors of GCN5-related N-acetyltransferases (GNATs) family, including GCN5 and p300/CBP-associating factor (PCAF) and MYST superfamily such as Garcinol, PU141, C646, Tip60 inhibitors TH1834, NU9056, and 6-alkylsalicylates, and BRD inhibitors such as I-BET 151, I-BET 762, OTX015, MK-8628, birabresib

In some instances, therapties may be used in combination, such as an anti-EGFR therapy and an anti-EGFR therapy. Anti-EGFR therapy may be used in combination with any combination of chemotherapeutic agents or chemotherapeutic regimens, for example, FOLFOX (fluorouracil [5-FU]/leucovorin/oxaliplatin), FOLFIRI (5-FU/leucovorin/irinotecan), and the like. Further information is found in Wu et al., “Small Molecules Targeting HATs, HDACs, and BRDs in Cancer Therapy” Front Oncol. 2020; 10:560487, which is fully incorporated by reference herein.

In some aspects, a cancer treatment ai administered to a subject. In some cases, the cancer treatment is administered in combination another therapy, such as a non-anti-EGFR therapy with anti-EGFR therapy.

Of note, pseudogenes can abolish the pathogenic effects of miRNAs or elements with microRNA response elements (MREs), such as RNA binding proteins (RBPs). Additionally, pseudogenes structure confers competitive interaction with miRNAs, indicating a parallel expression pattern between the pseudogene and the miRNA. Honology to miRNA target genes can restrain pathogenic effects by producing antisense RNAs or siRNAs. Notably, pseudogenes can generate several lncRNAs and one can decipher pseudogene activity and their sequences to create more pseudogene analogs that better facilitate production of miRNA decoy, or in other embodiments, antisense RNA, siRNA and lncRNA producer functions, may be an ideal strategy. In other embodiments, transcribed pseudogenes are utilized as antigens to activate the innate or adaptive immune response in the human body. In other embodiments, transcribed pseudogenes can be redesigned to produce peptides with high antigenicity and immunogenicity.

### Genetic Analysis

Genetic analysis includes detection of nucleotide sequence variants and copy number variation, pseudogenes, number of pseudogenes, retrotransposon activity. Genetic variants can be determined by sequencing. The sequencing method can be massively parallel sequencing, that is, simultaneously (or in rapid succession) sequencing any of at least 100,000, 1 million, 10 million, 100 million, or 1 billion polynucleotide molecules. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next-generation sequencing, Single Molecule Sequencing by Synthesis (SMSS) (Helicos), massively-parallel sequencing, Clonal Single Molecule Array (Solexa), shotgun sequencing, Maxam-Gilbert or Sanger sequencing, primer walking, sequencing using PacBio, SOLID, Ion Torrent, or Nanopore platforms and any other sequencing methods known in the art.

Sequencing can be made more efficient by performing sequence capture, that is, the enrichment of a sample for target sequences of interest, e.g., sequences including the KRAS and/or EGFR genes or portions of them containing sequence variant biomarkers. Sequence capture can be performed using immobilized probes that hybridize to the targets of interest.

Cell free DNA can include small amounts of tumor DNA mixed with germline DNA. Sequencing methods that increase sensitivity and specificity of detecting tumor DNA, and, in particular, genetic sequence variants and copy number variation, pseudogenes, number of pseudogenes, retrotransposon activity, can be useful in the methods of this invention. Such methods are described in, for example, in WO 2014/039556. These methods not only can detect molecules with a sensitivity of up to or greater than 0.1%, but also can distinguish these signals from noise typical in current sequencing methods. Increases in sensitivity and specificity from blood-based samples of cfDNA can be achieved using various methods. One method includes high efficiency tagging of DNA molecules in the sample, e.g., tagging at least any of 50%, 75% or 90% of the polynucleotides in a sample. This increases the likelihood that a low-abundance target molecule in a sample will be tagged and subsequently sequenced, and significantly increases sensitivity of detection of target molecules.

Another method involves molecular tracking, which identifies sequence reads that have been redundantly generated from an original parent molecule, and assigns the most likely identity of a base at each locus or position in the parent molecule. This significantly increases specificity of detection by reducing noise generated by amplification and sequencing errors, which reduces frequency of false positives.

Methods of the present disclosure can be used to detect genetic variation in non-uniquely tagged initial starting genetic material (e.g., rare DNA) at a concentration that is less than 5%, 1%, 0.5%, 0.1%, 0.05%, or 0.01%, at a specificity of at least 99%, 99.9%, 99.99%, 99.999%, 99.9999%, or 99.99999%. Sequence reads of tagged polynucleotides can be subsequently tracked to generate consensus sequences for polynucleotides with an error rate of no more than 2%, 1%, 0.1%, or 0.01%.

Copy number variation, pseudogenes, number of pseudogenes, retrotransposon activity, determination can involve determining a quantitative measure of polynucleotides in a sample mapping to a genetic locus, tumor promoter genes include ADAM5, ACTG1P25, AK4P1, BRAFP1, BRCA1P1, CYP2A7, CYP4Z2P, DUXAP8, EBLN3P, FTH1P3, FLT1P1-S, OGFRP1, LGMNP1, MSTO2P, MYLKP1, OCT4-pg4, PCNAP1, PDIA3P1, PPM1K, PRELID1P6, PTENP1-AS, PTTG3P, RPSAP52, SALL4P5, TCAM1P, TDGF1P3, RP9P, UBE2CP3, or tumor suppressor genes such as ARHGAP27P1, FLT1P1-AS, FOXO3P, Pseudogenes of FTH1, GUSBP11, MT1JP, PEBP1P2, SNRPFP1, SNX17 and TUSC2P (adapted from Han et. al. (2014)) or a gene set forth in Table 1 (adapted from Nakamura-García (2023)). The quantitative measure can be a number. Once the total number of polynucleotides mapping to a locus is determined, this number can be used in standard methods of determining Copy number variation, pseudogenes, number of pseudogenes, retrotransposon activity, at the locus. A quantitative measure can be normalized against a standard. In one method, a quantitative measure at a test locus can be standardized against a quantitative measure of polynucleotides mapping to a control locus in the genome, such as gene of known copy number. In another method, the quantitative measure can be compared against the amount of nucleic acid in the original sample. For example, the quantitative measure can be compared against an expected measure for diploidy. In another method, the quantitative measure can be normalized against a measure from a control sample, and normalized measures at different loci can be compared. In another method, quantifying involves quantifying parent or original molecules in a sample mapping to a locus, rather than number of sequence reads. A copy number variation, pseudogenes, number of pseudogenes, retrotransposon activity, may be an amplification or a deletion or truncation of a gene. An amplification may be 3, 4, 5, 6, 7, 8, 9, 10, or 10 or more copies of a gene. A deletion or truncation may be 0 or 1 copies of a gene.

An example of a method for detecting copy number variation, pseudogenes, number of pseudogenes, retrotransposon activity, may include an array. The array may comprise a plurality of capture probes. The capture probes can be oligonucleotides that are bound to the surface of the array. The capture probes may hybridize to ADAM5, ACTG1P25, AK4P1, BRAFP1, BRCA1P1, CYP2A7, CYP4Z2P, DUXAP8, EBLN3P, FTHIP3, FLT1P1-S, OGFRP1, LGMNP1, MSTO2P, MYLKP1, OCT4-pg4, PCNAP1, PDIA3P1, PPM1K, PRELID1P6, PTENP1-AS, PTTG3P, RPSAP52, SALL4P5, TCAM1P, TDGF1P3, RP9P, UBE2CP3, or ARHGAP27P1, FLT1P1-AS, FOXO3P, Pseudogenes of FTH1, GUSBP11, MT1JP, PEBP1P2, SNRPFP1, SNX17 and TUSC2P. The capture probes may bind to at least 1, 2, 3, 4, 5, 6, 7, 8, 9, 10, 11, or 12 genes. DNA derived from the subject may be labeled (e.g., with a fluorophore) prior to hybidization for detection.

In other examples, a gene of interest may be amplified using primers that recognize the gene of interest. The primers may hybridize to a gene upstream and/or downstream of a particular region of interest (e.g., upstream of a mutation site). A detection probe may be hybridized to the amplification product. Detection probes may specifically hybridize to a wild-type sequence or to a mutated/variant sequence. Detection probes may be labeled with a detectable label (e.g., with a fluorophore). Detection of a wild-type or mutant sequence may be performed by detecting the detectable label (e.g., fluorescence imaging). In examples of copy number variation, pseudogenes, number of pseudogenes, retrotransposon activity, a gene of interest may be compared with a reference gene. Differences in copy number between the gene of interest and the reference gene may indicate amplification or deletion/truncation of a gene. Examples of platforms suitable to perform the methods described herein include digital PCR platforms such as e.g., Fluidigm Digital Array.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

Long INterspersed Element 1 (LINE-1) retrotransposon, mobilizes through retrotransposition. This copy and paste mechanism utilizes an RNA intermediate, and LINE-1 copies account for over 17% of the modern human DNA. Full length LINE-1 sequences consist of a 5′UTR/promoter, two open reading frames, coding for ORF1p and ORF2p proteins, and a 3′ UTR with a polyA signal. Following transcription by RNA polymerase II, LINE-1 mRNA is exported from the nucleus. ORF1p and ORF2p are translated in the cytoplasm, bind LINE-1 mRNA and form LINE-1 ribonucleoproteins (RNP) composed of LINE-1 mRNA coated by ORF1p trimers and presumably one or a few ORF2p.

In cells undergoing division, LINE-1 RNPs can enter the nucleus upon mitotic nuclear membrane breakdown. ORF2p nicks the DNA in A/T rich regions (AA/TTTT consensus) using its endonuclease domain, leading to insertion of a new copy of LINE-1 through its reverse transcriptase domain. LINE-1 demonstrates strong cis preference in mobilizing its own mRNA, but its proteins have also been shown to bind and mobilize non-LINE-1 mRNA such as SINEs and other mRNAs that produced processed pseudogenes.

DNA methylation, histone modifications, and RNA interference all limit LINE-1 expression and function in somatic cells. Without being bound by any particular theory, it appears mechanisms that limit LINE-1 expression are often dysfunctional in cancers, allowing for the expression and mobilization of LINE-1. LINE-1 expression has the potential to disrupt genomic stability, making it a likely component in cancer progression. Nevertheless, the repetitiveness of LINE-1 sequences poses a challenge to identifying actively transcribed LINE-1 loci.

### Example 2

Some LINE-1 detection techniques quantify LINE-1 transcripts using an expectation maximization algorithm method that is similar to methods used to quantify gene isoforms. For example, LIEM model starts with a list of transcripts that includes each of the possible transcript types at each locus. LIEM then calculates the extent to which each read supports each transcript and fractionally assigns reads to transcripts based on this support. The fractional assignments provide an initial estimate of the relative transcript abundances, which can be used to refine the fractional assignments, which in turn are used refine the transcript abundance estimates. These steps are repeated until convergence.

### Example 3

In addition, the described methods and compositions can be applied to all cancer therapies. Methylation is a critical gene regulatory mechanism and it is expected that any biological perturbation will result in changes in the methylome. Such changes can be analyzed and interpreted to determine the biological response, at the epigenomic gene regulation level, to assist in predicting the long-term clinical outcome of the patient.

More specifically, the described methods and compositions can be applied to cancer therapies with a mechanism of action known to clearly impact methylation. Such therapies include HDAC inhibitors such as vorinostat, romidepsin, panobinostat, chidamide and belinostat HAT inhibitors such as inhibitors of GCN5-related N-acetyltransferases (GNATs) family, including GCN5 and p300/CBP-associating factor (PCAF) and MYST superfamily such as Garcinol, PU141, C646, Tip60 inhibitors TH1834, NU9056, and 6-alkylsalicylates, and BRD inhibitors such as I-BET 151, I-BET 762, OTX015, MK-8628, birabresib These therapies alter epigenomic regulation and cause changes in the methylome.

### Example 4

In addition, the described methods and compositions can be applied to cancer therapies with a direct impact on methylation, for instance methylating agents such as TMZ and dacarbazine and demethylating agents such as azacytidine and decitabine. The resulting methylation changes directly reflect the molecular success, or lack thereof, that these agents are able to induce in the tumor.

Important features of the method are multiple timepoints of blood-based testing, detection of genomic and methylomic changes in these samples, and analysis of the biological functions of the gene(s) impacted by such methylation.

### Example 5

The described methods and compositions can be supplemented by additional analyses such as transcriptomic and proteomic changes. These changes reflect additional dimensions in which the patient and/or the tumor is responding to cancer therapy.

Furthermore, methylation is involved in multiple diseases and changes in the methylome after treatment initiation may predict long-term clinical response outside of oncology.

### Example 6

Described are methods including determining a plurality pseudogene sequences, determining a plurality of sequence reads of a target region of a chromosome of a subject, wherein the target region of the chromosome comprises one or more loci, aligning the plurality of sequence reads to the plurality of known or suspected pseudogene allele sequences, determining, based on the alignment, for each known or suspected pseudogene allele sequence of the plurality of known or suspected pseudogene allele sequences, a number of sequence reads that aligned to each known or suspected pseudogene allele sequence, and determining, based on the numbers of sequence reads that aligned to each known pseudogene or suspected allele sequence, for the one or more loci, the known allele sequences present at the one or more loci. Further, the method includes determining retrotransposition activity based on the known pseudogene allele sequences present at the one or more loci.

### Example 7

Described are methods including determining a plurality of pseudogene allele sequences by determining a plurality of sequence reads of a target region of a chromosome of a subject, wherein the target region of the chromosome comprises one or more loci including known or suspected psudeogene allele sequences, aligning the plurality of sequence reads to the plurality of known pseudogene allele sequences, determining, based on the alignment, for each known or suspected psudeogene allele sequence of the plurality of known or suspected pseudogene allele sequences, a number of sequence read families (i.e., number of nucleic acid molecules—a sequence read family may be a group of sequence reads corresponding to a single nucleic acid molecule) that aligned to each known pseudogene allele sequence, and determining, based on the numbers of sequence read families that aligned to each known pseudogene allele. Further, the method includes determining retrotransposition activity based on the known pseudogene allele sequences present at the one or more loci.

### Example 8

Described herein are method including determining a plurality of sequence reads of a target region of a chromosome of a subject, wherein the target region of the chromosome comprises one or more loci, subtracting concordantly mapped pairs corresponding to reference genome including reference repeats, aligning to pre-built database of repeats, identifying read pairs where only one read is mapped to a repeated, extracting unmapped reads, realigning unmapped reads to reference genome, identifying sets of new integration sites. Further, the method includes determining retrotransposition activity based on the known allele sequences present at the one or more loci.

### Example 9

Retrotransposon activation in tumors may contribute to their development into inflamed and T-cell-infiltrated tumors. Cancer therapeutics and chemotherapeutic agents have been shown to activate retrotransposon expression in cancer. Cyclin-dependent kinases 4 and 6 (CDK4/6) inhibitors repressed DNMT1 and caused activation of repeat elements, including retrotransposons in breast cancer. cells could modulate retrotransposon expression with lethal drug exposures by maintaining their epigenetic repression. As such, the aforementioned methods could characterize the cancer afflicting a subject, thereby providing a effective course of treatment, such as for example, combined used of agents such as multiple types of epigenetic inhibitors, or an epigenetic inhibitor with another targeted therapy.

1. A method comprising:
determining a plurality of sequence reads of a target region of a chromosome of a subject, wherein the target region of the chromosome comprises one or more loci;
aligning the plurality of sequence reads to a reference genome;
subtracting aligned pairs from the plurality of sequence reads to generate a plurality of candidate sequence reads;
aligning the plurality of candidates sequences reads to a plurality of known allele sequences;
determining, based on the alignment, for each known allele sequence of the plurality of known allele sequences, a number of sequence reads that aligned to each known allele sequence;
extracting unaligned candidate sequence reads;
mapping unaligned candidate sequence reads to the reference genome; and
determining, based on the unaligned candidate sequence reads that mapped to the reference genome, one or more integration sites present at the one or more loci.

2. The method of claim 1, wherein the plurality of known allele sequences comprise a plurality of known reference repeats.

3. The method of claim 1, wherein only one read of a read pair is aligned to a repeat.

4. The method of method of claim 1, comprising determining retrotransposition activity based on the one or more integration sites present at the one or more loci.

5. The method of claim 1, further comprising:
obtaining a sample from the subject; and
sequencing the sample to obtain the plurality of sequence reads of the target region of the chromosome.

6. The method of claim 1, further comprising:
determining, based on the mapping, for each read of the plurality of sequence reads, one or more integration sites present at the one or more loci.

7. The method of claim 1, wherein the target region comprises one or more of the following genes: ARHGAP27P1, FLT1P1-AS, FOXO3P, Pseudogenes of FTH1, GUSBP11, MT1JP, PEBP1P2, SNRPFP1, SNX17 and TUSC2P.

8. The method of claim 1, wherein the target region comprises one or more of the following genes: ADAM5, ACTG1P25, AK4P1, BRAFP1, BRCA1P1, CYP2A7, CYP4Z2P, DUXAP8, EBLN3P, FTH1P3, FLT1P1-S, OGFRP1, LGMNP1, MSTO2P, MYLKP1, OCT4-pg4, PCNAP1, PDIA3P1, PPM1K, PRELID1P6, PTENP1-AS, PTTG3P, RPSAP52, SALL4P5, TCAM1P, TDGF1P3, RP9P, UBE2CP3.

9. The method of claim 1, wherein determining one or more integration sites comprises identifying one or more exon-exon junctions in the reference genome.

10. The method of claim 9, wherein the one or more exon-exon junctions is not of germline origin.

11. The method of claim 9, wherein determining one or more integration sites comprises mapping to a maximum pseudogene sequence comprising all exons.

12. The method of claim 11, wherein mapping comprises determining unaligned candidate sequence spanning one or more exon-exon junctions.

13. The method of claim 12, wherein two reads of a read pair is mapped to different exons.

14. The method of claim 1, wherein determining, based on the numbers of sequence reads that aligned to each known allele sequence, for the one or more loci, the known allele sequences present at the one or more loci comprises determining one or more known allele sequences having a highest number of sequence reads aligned.

15. The method of claim 1, wherein the reads that aligned to each known allele sequence are grouped into read families, and the method further comprises, determining a number of sequence read families that are aligned to each known allele sequence.

16. The method of claim 12, wherein determining, for the one or more loci, the known allele sequences present at the one or more loci based on the numbers of sequence read families that aligned to each known allele sequence.

17. The method of claim 1, further comprising determining a length of a portion of each known allele sequence aligned to two or more sequence reads of the plurality of sequence reads.

18. The method of claim 1, further comprising:
sorting, for a locus, the known allele sequences present at the locus by the number of sequence reads that aligned to each known allele sequence;
determining, for the locus, a first known allele sequence with a highest number of sequence reads aligned;
inserting the first known allele sequence with the highest number of sequence reads aligned into a superset;
determining one or more known allele sequences that aligned to reads that are a subset of the reads aligned to the first known allele sequence; and
inserting the one or more known allele sequences into the superset.

19. The method of claim 15, wherein the superset comprises a graph data structure.

20. The method of claim 16, wherein the graph data structure comprises a directed acyclic graph.

21. The method of claim 16, wherein the graph data structure represents a Hasse diagram.

22. The method of claim 15, further comprising:
determining that the locus is associated with a single superset; and
determining the first known allele sequence of the single superset as the allele present at the locus.

23. The method of claim 15, further comprising determining a plurality of supersets for the locus.

24. The method of claim 20, further comprising
determining, based on the plurality of supersets for the locus, two supersets with a cumulative largest number of distinct reads; and
determining the first known allele sequence of each of the two supersets as the alleles present at the locus.

25. The method of claim 1, further comprising, assisting in a communication of the known allele sequences present at the one or more loci to a medical provider.
